(19) United States
(12) Patent Application Publication
(10) Pub. No.: US 2026/0000880 A1
(43) Pub. Date: Jan. 1, 2026

(54) USE OF CAVITATION INDUCED BY PULSED FOCUSED ULTRASOUND TO ALTER TISSUE MICROENVIRONMENT

(71) Applicant: University of Washington, Seattle, WA (US)

(72) Inventors: Tatiana D. KHOKHLOVA, Seattle, WA (US); Minho SONG, Seattle, WA (US); Randall WILLIAMS, Seattle, WA (US); Gilles Pierre Loic THOMAS, Seattle, WA (US); Yak-Nam WANG, Seattle, WA (US); Vera A. KHOKHLOVA, Seattle, WA (US)

(73) Assignee: University of Washington, Seattle, WA (US)

(21) Appl. No.: 19/247,337

(22) Filed: Jun. 24, 2025

Related U.S. Application Data
(60) Provisional application No. 63/665,602, filed on Jun. 28, 2024.

(51) Int. Cl. A61M 37/00; A61B 8/00; A61B 8/14

(52) U.S. Cl. CPC A61M 37/0092; A61B 8/4488; A61B 8/14; A61M 2205/3375

(57) ABSTRACT

A pulsed focused ultrasound (pFUS) system, including a dual mode ultrasound probe comprising a plurality of ultrasound emitting elements, wherein a first group of elements of the plurality of elements is configured to generate therapy ultrasound bursts, and wherein a subset of the first group of elements is configured to generate imaging ultrasound bursts. Further, a method for tissue treatment in a subject using pulsed focused ultrasound (pFUS), including operating a dual-mode ultrasound probe as a therapy ultrasound probe and operating the dual-mode ultrasound probe as an imaging ultrasound probe where the dual-mode ultrasound probe is configured for generating the therapy ultrasound bursts and the imaging ultrasound bursts by same ultrasound emitting elements.

### CROSS-REFERENCE TO RELATED APPLICATION

This application claims the benefit of U.S. Provisional Application 63/665,602, filed Jun. 28, 2024, the entire disclosure of which is incorporated herein.

### STATEMENT OF GOVERNMENT LICENSE RIGHTS

This invention was made with government support under Grant Nos. 2R01CA154451 and R01EB023910, awarded by the National Institutes of Health. The government has certain rights in the invention.

### BACKGROUND

Ultrasound contrast agents (UCAs), such as microbubbles and nanodroplets, have been successfully used in combination with acoustic cavitation for targeted drug delivery. In one approach, UCAs are intravenously administered and travel through the circulatory system to the target location and can be monitored using ultrasound imaging. Upon reaching the target, the UCAs can then be activated by applying ultrasound of sufficient amplitude to rupture the UCAs, thereby either releasing their payload or affecting the vasculature and/or perivascular tissue permeability. All of the above effects enhance diffusion of co-administered or subsequently administered drug to the targeted tissue. While the approach has been studied and successfully used over the past decade, many types of tumors, such as pancreatic ductal adenocarcinoma (PDAC) and liver metastases thereof, present barriers to targeted anticancer drug delivery, such as dense stroma, high interstitial fluid pressures, and irregular vascularization. As microbubbles are confined to blood vessels and perivascular space, this greatly reduces the efficacy of UCAs for targeted drug delivery in PDAC.

Accordingly, devices and methods for improved targeted drug delivery are needed.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, disclosed herein is a pulsed focused ultrasound (pFUS) system, including a dual mode ultrasound probe configured to generate therapy ultrasound bursts and generate imaging ultrasound bursts from a same one or more ultrasound emitting elements.

In some embodiments, the pFUS system is a dual phased array, and wherein the one or more ultrasound emitting elements is a plurality of ultrasound emitting elements. In some embodiments, a first group of elements of the plurality of elements is configured for generating therapy ultrasound bursts, and wherein a subset of the first group of elements is configured for generating imaging ultrasound bursts.

In some embodiments, the pFUS system is configured for operating the dual-mode ultrasound probe as a therapy ultrasound probe by: by the plurality of ultrasound emitting elements, generating millisecond-long therapy ultrasound bursts that are nonlinearly distorted at a beam focus area of a tissue, in response to the therapy ultrasound bursts, generating bubble cavitation in the tissue at the beam focus area, mechanically disrupting the tissue by the bubble cavitation, by disrupting the tissue, increasing permeability of the tissue to a drug administered to the subject, and by increasing permeability, enhancing passive diffusion of the drug.

In some embodiments, the pFUS system is configured for operating the dual-mode ultrasound probe as an imaging ultrasound probe by adjusting pressure levels at the focus area by adjusting parameters of the ultrasound probe, and by the subset of the first group of elements, generating imaging ultrasound bursts at the beam focus area of the tissue.

In some embodiments, the tissue is disrupted non-thermally. In some embodiments, the tissue comprises cellular r membranes, collagen matrices, or glycosaminoglycan GAG-water complexes.

In some embodiments, the pFUS array system further comprises a controller, wherein the controller is configured for adjusting the power supply voltage amplitude and frequency, or varying the duty cycle of the stimulus waveform, or a pulse duration of the ultrasound therapy.

In some embodiments, the imaging ultrasound bursts are B-mode imaging.

In another aspect, disclosed herein is a pulsed focused ultrasound (pFUS) array system for ultrasound imaging-guided treatment, including a multi-element linear array operable at a frequence range of 1-1.5 MHz and having an aperture with dimensions of approximately 14.8 mm×51.2 mm, a cylindrical lens configured to focus the linear array to depth in tissue of approximately 50 mm, and driving electronics with electronic beam steering capabilities and power levels configured to drive the linear array sufficient to achieve the focal pressures in tissue producing focal waveforms with shock fronts up to 45 MPa and peak negative pressures up to 9 MPa at focusing distances of 38-75 mm from the array.

In yet another aspect, disclosed herein is a method for tissue treatment in a subject using pulsed focused ultrasound (pFUS), the method including operating a dual-mode ultrasound probe as a therapy ultrasound probe by generating millisecond-long therapy ultrasound bursts that are nonlinearly distorted at a beam focus area of the tissue, in response to the therapy ultrasound bursts, generating bubble cavitation at the beam focus area, mechanically disrupting the tissue by the bubble cavitation, by disrupting the tissue, increasing permeability of the tissue to a drug administered to the subject, and by increasing permeability, enhancing passive diffusion of the drug, operating the dual-mode ultrasound probe as an imaging ultrasound probe by adjusting pressure levels at the focus area by adjusting parameters of the ultrasound probe, and generating imaging ultrasound bursts at the beam focus area of the tissue, where the dual-mode ultrasound probe is configured for generating the therapy ultrasound bursts and the imaging ultrasound bursts by same ultrasound emitting elements.

In some embodiments, the method further includes, administering the drug to the subject. In some embodiments, the tissue is disrupted non-thermally. In some embodiments, the tissue comprises collagen matrices, or glycosaminoglycan GAG-water complexes.

In some embodiments, the method further includes adjusting the power supply voltage amplitude and frequency, or varying the duty cycle of the stimulus waveform, or a pulse duration. In some embodiments, the method further includes administering chemotherapy drug to the patient after disrupting the tissue.

In some embodiments, the dual-mode ultrasound probe is a phased array probe, and wherein the dual-mode ultrasound probe is configured for generating the therapy ultrasound bursts by a first group of elements, and generating the imaging ultrasound bursts by a subset of the first group of elements.

In some embodiments, the imaging ultrasound bursts is B-mode imaging.

In some embodiments, the pFUS transducer array system further comprises a targeting and cavitation monitoring component.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a schematic diagram of an ultrasound system in accordance with an embodiment of the present technology.

FIG. 2 is an example dual-mode ultrasound array, in accordance with the present technology.

FIG. 3 is another example dual-mode ultrasound array, in accordance with the present technology.

FIG. 4 is an example array of ultrasound emitting elements, in accordance with the present technology.

FIGS. 5A-5B show beam profiles, measured through the focus, when electronic phasing is used to focus the beam along the Z axis (a) and for azimuthal steering along the X axis (b), in accordance with the present technology.

FIGS. 6A-6F show the source pressure magnitude and phase distributions, reconstructed from the measured holograms, when the array is configured for no electronic focusing on-axis focusing at 50 mm and focusing at 50 mm and steering towards 10 mm in accordance with the present technology.

FIG. 7 is a graph of the comparison of measured focal pressure waveforms and nonlinear simulation results based on the reconstructed boundary condition for the nominal focus position, at three different stimulus levels, in accordance with the present technology.

FIGS. 8A-8C show measured nonlinear focal pressures and shock amplitudes as a function of stimulus voltage for focal distances of 38 mm, 50 mm, and 75 mm without transverse beam steering compared with results from nonlinear simulation based on the holographic boundary conditions, in accordance with the present technology.

FIG. 9 shows peak pressures for the nominal steering configuration and for beam steering to XF=10 mm and ZF=50 mm, vs. applied stimulus voltage, in accordance with the present technology.

FIGS. 10A-10D are high-speed camera images showing the distribution of induced cavitation bubbles in the focal region, for on-axis beam focusing at ZF=50 mm, in accordance with the present technology.

FIG. 11 shows categories of cavitation behavior observed in high-speed images for peak negative focal pressures between 1 MPa and 9 MPa, for on-axis focusing with focal lengths of 38 mm, 50 mm, and 75 mm, in accordance with the present technology.

FIGS. 12A-12B are contour plots showing the peak negative pressure distribution (top) and peak positive (bottom) near the beam focus in the azimuthal plane, computed by nonlinear simulation with the source boundary condition measured by holography, in accordance with the present technology.

FIGS. 13A-13F are B-Mode images acquired with the dual-mode probe and Verasonics V-1 system at 1.5 MHz, compared with those collected using a 6C2s probe and BK3000 scanner at 2.5 MHz in accordance with the present technology.

FIGS. 14A-14C are B-mode image of a single wire target in the CIRS phantom with a diameter of 100 μm and at an axial depth of approximately 4.7 cm, the intensity profile extracted through the center of the target image, and the pressure waveform measured at the focus of the imaging beam, respectively, in accordance with the present technology.

FIG. 15 is an example method of operating a dual phase probe as described herein, in accordance with the present technology.

### DETAILED DESCRIPTION

Pulsed focused ultrasound (pFUS) uses nonlinearly distorted millisecond-long ultrasound pulses of moderate intensity to induce inertial cavitation in tissue without administration of contrast agents. Depending on the pressure levels and degree of waveform nonlinearity, different cavitation behaviors can be achieved with different resulting mechanical disruption of tissue, from grossly visible areas of complete fractionation to mild transient changes that are only seen histologically. This can be used for a number of clinical applications. Stronger cavitation behaviors can be used to non-invasively permeabilize tissue, such as tumor tissue, to improve passive diffusion of systemically administered drugs. This can be particularly useful for tumors that are very dense and fibrous, with high concentration of glucosaminoglycans (GAGs), such as pancreatic tumors. Tumor permeabilization can include disruption of collagen matrix and GAG-water complexes and damage to cellular membranes. In a mouse model of pancreatic cancer, pFUS-induced cavitation combined with chemotherapy has been shown to have a pronounced tumoricidal effect, converting the tumor microenvironment to be more immunogenic, and reducing tumor chemoresistance. Another application of cavitation-based disruption of collagen and GAGs is softening of nucleus pulposus of the intervertebral disks (IVDs) and IVD hernias for subsequent removal or to reduce the associated pressure on the nerves. Milder cavitation behaviors can be used to induce inflammatory signaling in tissue without grossly observable damage. One application is promoting muscle growth and vascularization for treatment of muscular dystrophy caused by disuse. To achieve those effects in a volume of tissue pFUS transducer arrays with a small size and footprint comparable to conventional US imaging probes can be used. Such a pFUS array that can be used for both cavitation-based therapy and US imaging for targeting and cavitation monitoring is disclosed herein.

As described above, pulsed high-intensity focused ultrasound (pHIFU) uses nonlinearly distorted millisecond-long ultrasound pulses of moderate intensity to induce inertial cavitation in tissue without administration of contrast agents. The resulting mechanical disruption permeabilizes the tissue and enhances the diffusion of systemically administered drugs. This is especially beneficial for tissues with poor perfusion, such as pancreatic tumors. Disclosed herein is a dual-mode ultrasound array designed for image-guided pHIFU therapies in producing inertial cavitation and ultrasound imaging. We focus on practical implementation and performance characterization of a dual-mode pHIFU array for cavitation-based tissue permeabilization in the context of enhancing image-guided drug delivery in PDAC. In some embodiments, using a single probe for both targeting via B-mode imaging and generating cavitation has significant advantages over using a focused therapy array combined with a coaxial imaging array. First, using a single probe eliminates the need for axial alignment of the therapeutic beam and imaging beam. Second, the use of a spherically-focused HIFU transducer results in a beam which has high-intensity components propagating in three full dimensions while conventional imaging probes only image in a two-dimensional plane. As a result, configuration of the conventional probe does not allow observing the entire three-dimensional region being insolated by the therapeutic beam. In contrast with the conventional technologies, using a dual-mode array for both targeting and application of pHIFU ensures that any anatomical feature insolated by the probe is also visible in the B-mode image, so that it is easier during clinical applications to avoid applying HIFU to sensitive or highly reflective structures such as bones or gaseous regions in the digestive tract. Stated differently, by using the same probe for the focused therapy and for the imaging, the inventive technology assures that the therapy area and the focal area are collocated.

In terms of acoustic output, the electronic steering range in the linear regime, defined here as the peak focal pressure being within 10% of that without steering, was measured and compared to the expectations from design-stage numerical modeling. The axial steering performance was in good agreement with the expectations, whereas the azimuthal steering limits are almost a factor of two lower than expected (11.5 mm vs. 22 mm). The most likely reason for this discrepancy is that the design-stage model assumed perfect cylindrical focusing in the elevational direction and did not account for refraction and imperfections in acoustic coupling of the array elements into the rubber lens. Additionally, the design-based model assumed that each element could be represented by a uniform rectangular velocity source. Any deviation of the actual velocity distribution at the face of an array element could modify the directivity of the element, which would have a subsequent impact on the overall steering performance of the array.

For azimuthally steered beams it was seen that similar nonlinear saturation curves result when the stimulus waveform amplitude is adjusted to account for the decrease in focal pressure due to steering in the linear operating regime. When the beam is steered azimuthally within the limits, the overall structure of the transmitted linear field remains similar to the nominal field. This justifies the use of azimuthal steering limits based on the 10% pressure variation for therapeutic applications where the focus of the beam is swept through the target region using electronic steering.

Regarding nonlinear acoustic behavior when the focal length is electronically varied, first, as the focal distance increases, the focal gain in the quasilinear operating regime is reduced due to the steering effect, as evidenced in the curves for both P₊ and P₋ at values of P₀ below approximately 0.5 MPa. Second, it was seen that less focused beams (i.e., beams with larger F-numbers) were able to generate shocks at the focus at lower power values and lower peak focal pressures. This occurs because the focal lobe is longer for less focused fields, and thus nonlinear effects accumulate over longer distances prefocally. The ability to electronically increase the focal length and F-number for the dual-mode probe allows for generating highly nonlinear pulses at decreasing peak negative pressures. The degree of nonlinearity and subsequent tissue disruption therefore can be tailored to the requirements of a given therapeutic application. This capability makes the probe potentially useful for applications beyond drug transport enhancement, such as using pHIFU to generate an inflammatory response or immune response with simultaneous B-mode targeting.

High-speed photography in agarose gel phantoms demonstrated the ability of the dual-mode array to induce de novo cavitation with characteristics necessary for efficient tissue permeabilization. At the nominal focal length of Z_{F}=50 mm, the threshold for inducing stationary cavitation at the focus corresponded to a peak negative pressure value of P_{31 =}2 MPa, while a transition to fan-out proliferating bubble behavior was observed at peak negative pressure value P₋₌5 MPa, when substantial nonlinear distortion and shock formation started. While for the case of Z_{F}=38 mm the transition to fan-out bubble proliferation occurred at a pressure below that required for shocks to form at the focus, the focal pressure waveforms still showed significant asymmetry due to nonlinearity, with peak positive pressure at the focus greater than the peak negative pressure by a factor of about two. Changing the focal length of the beam had a notable impact on the acoustic power required to generate proliferating cavitation at the focus, as the reflection of highly nonlinear pressure pulses from bubbles plays a major role in the formation of proliferating bubble clouds and nonlinear focal pressure waveforms occur at lower peak pressures for beams with larger F-numbers. The cavitation behaviors were categorized in this study based on the simple criteria of whether or not they were observed in any camera frame over three exposures at a particular power level and should thus be interpreted as minimum levels at which certain behaviors could be observed.

To illustrate the significance of both peak positive and peak negative pressure on the distribution of generated bubbles and transition to proliferating behavior, the bubble distribution directly captured using the high-speed camera for a maximum of P_{31 =}6 MPa in agarose was overlaid on contour plots of pressure near the focus. Stationary cavitation bubbles were seen to form throughout the region where P₋ exceeded the cavitation threshold of 2 MPa in the gel, while proliferating behaviors were only observed near the focus where the waveforms show a high degree of asymmetry due to nonlinear distortion and shock formation. Transport rates can increase with the intensity of induced bubble activity, as quantified using PCD signals. Proliferating bubbles move more vigorously than stationary bubbles do and are therefore expected to cause more tissue disruption than stationary bubbles. As a result, the effective treatment region will be smaller than is implied by the overall extent of the bubble distribution.

Regarding the potential for off-target tissue damage by stationary cavitation bubbles outside of the focal region, studies on the use of pHIFU for cardiac pacing have shown that de novo cavitation may be induced in tissues at peak pressures of up to 3 MPa without resulting in gross tissue damage.

While the B-mode images rendered using the dual-mode probe may lack the resolution of the clinical diagnostic imaging system, they are of sufficient quality for identifying centimeter sized targets and anatomical landmarks during pHIFU treatment. In the envisioned clinical scenario, a clinical ultrasound imaging system could be used to obtain high-quality images before treatment, and the dual-mode system could then be used to find anatomical landmarks for relative positioning of the pHIFU treatment area. The basic algorithm employed for B-mode imaging in this study, where a beam was swept across the imaging plane at a single focal depth, leaves room for additional image improvement using more advanced imaging techniques. For instance, multiple beams with different focal lengths and different center frequencies may be compounded in order to reduce speckle and improve image quality. In addition, nonlinear pulsing schemes such as pulse inversion and amplitude modulation harmonic imaging may be used to increase the imaging contrast by using the second harmonic of the transmitted pulse for image reconstruction. The limited bandwidth of 0.71 MHz for the dual-mode array prevented the use of harmonic imaging in this study, as the amplitudes of the received second harmonic signals were effectively filtered out by the inductive impedance matching network. Methods for improving the bandwidth of the system on the receiving side may enable harmonic imaging. Image quality may also be improved through the addition of a high-frequency transducer stack within the probe.

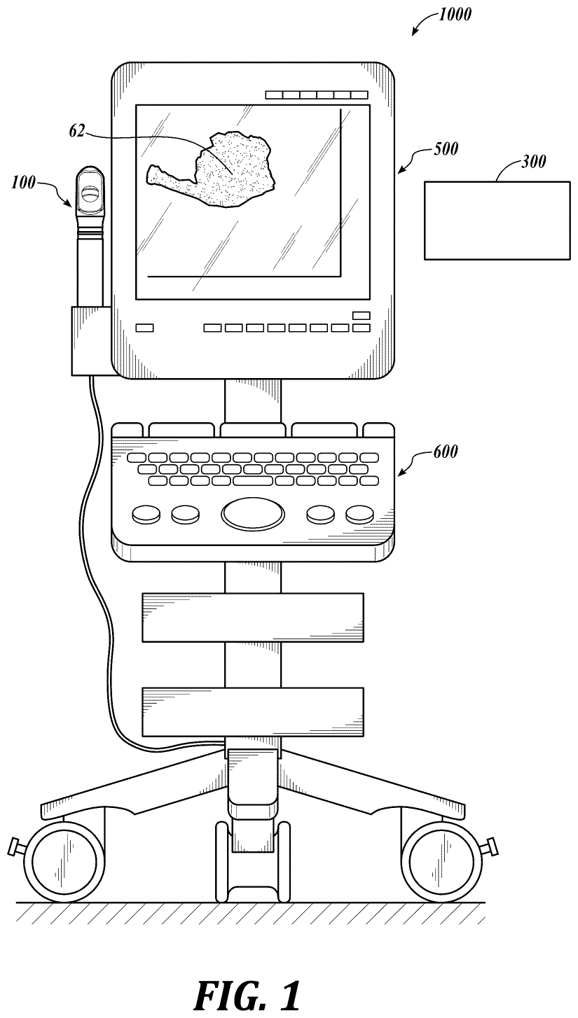

FIG. 1 is a schematic diagram of an ultrasound system 1000 in accordance with an embodiment of the present technology. The ultrasound system 1000 includes an ultrasound probe 100 having a therapy transducer and an imaging transducer. As explained above, the therapy transducer and imaging transducer may be the same element of the ultrasound probe that is sequentially used for either the therapy or the imaging. The ultrasound probe 100 may be controlled by a controller (e.g., a computer) 600 having suitable software and commands for controlling the ultrasound. A monitor 500 can display images 62 of the target tissue of the patient that are obtained, for example, by an imaging transducer of the ultrasound probe 100 or by a separate imaging device. In some embodiments, the ultrasound system 1000 may further include an external imaging device 300. In some embodiments, the external imaging device 300 is a clinical ultrasound imaging system configured to obtain high-quality images before treatment. In some embodiments, the external imaging device 300 is a fine ultrasound.

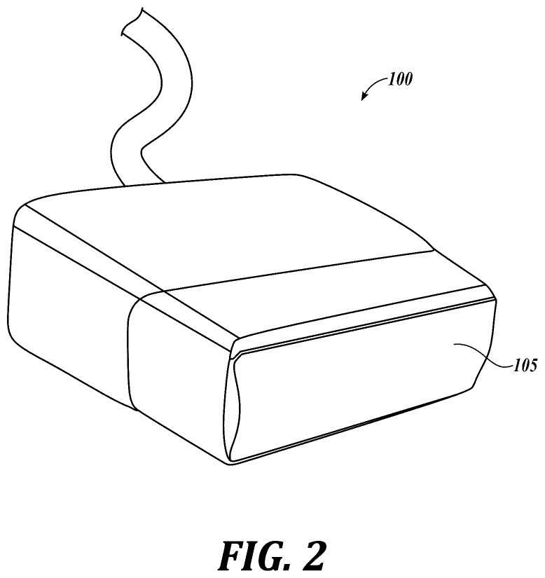

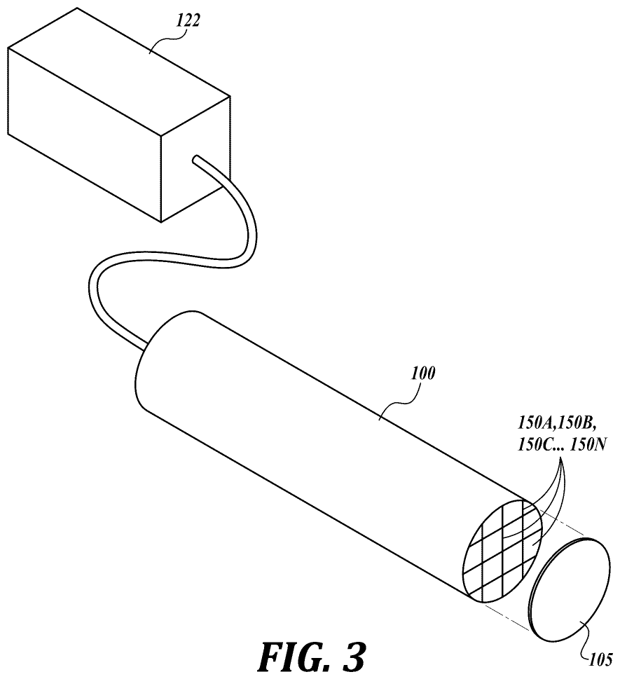

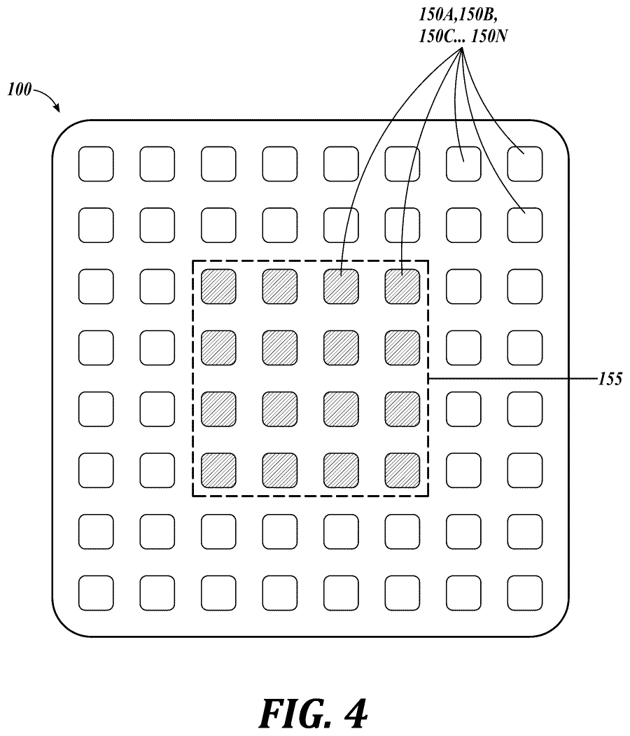

FIG. 2 is an example dual-mode ultrasound array, in accordance with the present technology. In one embodiment, shown in FIG. 2, the dual-mode ultrasound array has 64 piezocomposite elements (as shown in FIGS. 3-4) 14.8 mm tall and 0.7 mm wide, arranged in a linear array at a pitch of 0.8 mm, resulting in an overall aperture of 14.8 in height and 51.2 mm in width. A cylindrical silicone rubber lens provides elevational focusing with a nominal focal length of Z_{F}=50 mm.

The array can be driven by, for example, a Verasonics V-1 ultrasound system, configured with an extended burst option. Electronic phasing of the individual elements can provide focusing and steering along the azimuth. An exemplary, non-limiting center frequency for the array is 1.05 MHz, which represents a compromise between providing a capability of generating sufficient cavitation activity at a desirable tissue depth of 5 cm and providing the ability to produce B-mode ultrasound images of acceptable quality for guidance during therapy when operating near the upper limit of the matched bandwidth.

In some embodiments, the array can be connected to an inductive matching network, configured to present an input impedance to the V-1 system drive electronics of 100 ohms per element at a frequency of 1.05 MHz. To increase the current delivered to each transducer element, each element of the array can be connected through the matching network to two transmitters of the V-1 system driving the element in parallel. The V-1 system provides two methods for controlling the effective amplitude of the transducer stimulus voltage: directly adjusting the power supply voltage or varying the duty cycle of the stimulus waveform. Because the period of the driving voltage waveform must be an integer number of system clock cycles, the actual drive frequency for pulsed HIFU generation May be 1.071 MHz, slightly higher than the center frequency of the transducer. The center frequency used for imaging pulses can be 1.5 MHz, near the upper limit of the matched bandwidth of the transducer. In other embodiments, the dual-mode ultrasound transducer may be a monolithic transducer that sequentially operates in a therapy mode or an imaging mode.

FIG. 3 is another example dual-mode ultrasound array, in accordance with the present technology. In some embodiments, the probe 100 includes a plurality of ultrasound emitting elements 150A, 150B, 150C . . . 150N. In some embodiments, the probe 100 is communicatively coupled to a controller 122. The controller 122 may include driving electronics and control electronics. The lens 105 may be disposed over the plurality of ultrasound emitting elements 150A, 150B, 150C . . . 150N.

In operation, the controller 122 may adjust the power supply voltage amplitude and frequency, or vary the duty cycle of the stimulus waveform, or a pulse duration of the ultrasound therapy delivered by the probe 100. In some embodiments, the controller includes driving electronics with electronic beam steering capabilities and power levels configured to drive the linear array sufficient to achieve the focal pressures in tissue producing focal waveforms with shock fronts up to 45 MPa and peak negative pressures up to 9 MPa at focusing distances of 38-75 mm from the array. In some embodiments, the pFUS transducer array system further comprises a targeting and cavitation monitoring component, which may be part of the controller 122.

FIG. 4 is an example plurality of ultrasound emitting elements 150A, 150B, 150C . . . 150N, in accordance with the present technology. In some embodiments, the probe 100 includes a plurality of ultrasound emitting elements 150A, 150B, 150C . . . 150N, i.e., the probe 100 is a phased array probe. In some embodiments, the dual mode ultrasound probe 100 includes a plurality of ultrasound emitting elements 150A, 150B, 150C . . . 150N, wherein a first group of elements of the plurality of elements (in FIG. 4, all ultrasound emitting elements 150A, 150B, 150C . . . 150N) is configured to generate therapy ultrasound bursts, and wherein a subset of the first group of elements 155 is configured to generate imaging ultrasound bursts. In the context of this specification, the word subset designates a group of elements 155 out of those elements 150 that are used for generating the therapy ultrasound bursts. In some embodiments, the dual mode ultrasound probe 100 may be a monolithic ultrasound probe.

In operation, the dual-mode ultrasound probe 100 may operate as a therapy ultrasound probe by generating millisecond-long therapy ultrasound bursts that are nonlinearly distorted at a beam focus area of the tissue, in response to the therapy ultrasound bursts, generating bubble cavitation at the beam focus area, mechanically disrupting the tissue by the bubble cavitation, by disrupting the tissue, increasing permeability of the tissue to a drug administered to the subject; and by increasing permeability, enhancing passive diffusion of a drug. In some embodiments, the tissue is disrupted non-thermally, i.e., the tissue is not heated. In some embodiments, the drug is a chemotherapy drug.

Further, in operation, the dual-mode ultrasound probe 100 may operate as an imaging ultrasound probe by adjusting pressure levels at the focus area by adjusting parameters of the ultrasound probe and generating imaging ultrasound bursts at the beam focus area of the tissue. In some embodiments, the imaging ultrasound bursts are B-mode imaging, as explained in detail herein.

### Examples

A representative image of the dual-mode transducer is shown in FIG. 2. A sample linear ultrasound array was fabricated by Sonic Concepts, Inc., (Bothell, WA, USA) and comprises 64 piezocomposite elements, 14.8 mm tall and 0.7 mm wide with a pitch of 0.8 mm, resulting in an overall aperture of 51.2 mm by 14.8 mm. Electronic phasing of the individual elements provided focusing and steering along the azimuth, while a silicone rubber cylindrical lens provided elevational focusing with a nominal focal length of ZF=50 mm, corresponding to a typical depth for pancreatic tumors when imaged using ultrasound. A summary of relevant physical parameters for the array is given in Table 1. The use of lower frequencies has been shown to reduce the cavitation threshold and generate larger bubbles in agarose phantoms, while imaging resolution increases with higher center frequency. The design center frequency of 1.05 MHz was chosen as a compromise between being able to generate sufficient cavitation activity at a nominal tissue depth of 5 cm and the ability to produce B-mode ultrasound images of acceptable quality for guidance during therapy when operating near the upper limit of the matched bandwidth. The array was connected to an inductive matching network, designed to present an input impedance to the drive electronics of 100 ohms per element at a frequency of 1.05 MHz.

For the exemplary measurements, the probe was driven with a four-board Verasonics V-1 ultrasound research system (Verasonics, LTD., Kirkland, WA, USA), configured with an extended burst option, a 1200 W external power supply (Aim-TTI QPX600DP, Huntingdon, U. K.), and custom capacitor bank for generation of high-amplitude short HIFU pulses. To increase the current delivered to each transducer element, each element of the array was connected through the matching network to two transmitters of the V-1 system driving the element in parallel. The Verasonics system provides two methods for controlling the effective amplitude of the transducer stimulus voltage: directly adjusting the power supply voltage or varying the duty cycle of the stimulus waveform. Because the period of the driving voltage waveform must be an integer number of system clock cycles, the actual drive frequency for pulsed HIFU generation was 1.071 MHz, slightly higher than the center frequency of the transducer. The center frequency used for imaging pulses was 1.5 MHz, near the upper limit of the matched bandwidth of the transducer.

TABLE 1

| Parameter | Value | |
|---|---|---|
| Center frequency | 1.05 | MHz |
| Matched bandwidth (−3 dB) | 0.71 | MHz |
| Aperture | 51.2 mm × 14.8 mm | |
| Element count | 64 | |
| Element pitch | 0.8 | mm |
| Elevational focal distance, Z_{F} | 50 | mm |
| F-number (F/#) | 0.98/3.4 | |
| azimuthal/elevational | | |
| Max. electrical power, average | 320 | W |

To facilitate comparison of measurements for different beam configurations with nonlinear simulations, the transducer power output was measured as a function of the voltage supplied by the system to the matching network using the radiation force balance method, for focal lengths of 38 mm, 50 mm, and 75 mm. The angular spectrum approach was employed to calculate a correction factor for each beam configuration, using measured holograms. The total acoustic power was also used to compute an effective source pressure, P₀, calculated as:

$${P}_{0} = \sqrt{2 \frac{{ρ}_{0} {c}_{0} {W}_{0}}{{S}_{eff}}} , \qquad Equation 1$$

where W₀ is the acoustic power, ρ₀ and c₀ are the density and speed of sound in water, respectively, and S_{eff} is the effective surface area. Values were chosen to represent water at 20° C.: ρ₀=997.9 kg/m3, c₀=1482.4 m/s, and S_{eff}=(51.2 mm×14.8 mm)=758×10−6 m2.

Hydrophone measurements were performed in a water tank with an automated scanning system. The dual-mode ultrasound array was mounted to a fixed frame positioned above the tank with the face submerged in water to a depth of approximately 10 mm. The water was de-ionized using a custom filtration system and degassed to less than 20% dissolved O₂ using a permeable gas-liquid separation membrane. The hydrophone was connected with a mounting fixture to a computer-controlled three-axis positioner comprising stepper motors and linear slides from Velmex (Velmex Inc., Bloomfield, NY, USA) with a positioning resolution below 10 μm per step.

Three different hydrophones were used to accommodate a range of measured pressures. Measurements in the linear regime with peak negative pressures below 100 kPa were made using an Onda HGL-0200 lipstick hydrophone coupled to an AH-2020 preamplifier (Onda Corporation, Sunnyvale, CA, USA). A needle probe hydrophone with a 75 μm active element (Precision Acoustics model NH0075 with preamp model HP, Dorchester, Dorset, UK) was used for measuring peak pressures between 100 kPa and 1 MPa. Nonlinear focal pressure measurements were made using a fiber optic probe hydrophone (model FOPH 2000, RP Acoustics, Leutenbach, Germany).For the measurements of beam profiles and focal pressure waveforms in both linear and nonlinear regimes, the hydrophone voltage was captured by a digital oscilloscope (model MSOX3034T, Keysight Technologies, Santa Rosa, CA, USA), which was connected to a desktop computer running a custom Matlab (The MathWorks, Inc., Natick, MA, USA) program used for controlling the positioning system and data acquisition. During all hydrophone measurements, 64-cycle bursts were used, and peak pressures were reported as an average of at least 6 consecutive cycles within steady-state region of the pulse.

Nonlinear focal pressure waveforms were measured with FOPH as a function of transducer driving voltage within 1.6-58 V range for different focusing distances (38 mm, 50 mm, and 70 mm). The location of the focus was defined as the location of the largest peak positive pressure in shock-forming regime for a stimulus voltage of 40 volts (corresponding here to total peak acoustic power of 216 watts for the case of Z_{F}=50 mm). For drive voltages below 28 V, 32 waveforms were acquired and time-averaged, while 8 averages were used for voltages above 28 V. Various conventions for reporting the shock amplitude Pₛₕ can be found in the literature and differ by application. For example, in boiling histotripsy applications, a definition of shock amplitude was used based on the slope of the pressure waveforms, which was found to correlate well with calculated heat generation rates. For the purposes of this paper, where reflection of shocks from bubbles is more important than thermal effects, the reported shock amplitude effectively corresponds to the magnitude of the pressure jump at the shock front. Specifically, each of the experimentally measured focal pressure waveforms was inspected, and if the jump in pressure was more than 9 MPa over a period of 12.5 ns or less (four sample periods at a rate of 320 MHz), that was understood to constitute an ultrasound shock. When making comparisons between measured and simulated waveforms, the third derivative of the simulated pressure waveforms was calculated in order to find the point along the waveform where the change in curvature was the largest, and this was taken to be the start of the shock. Because of noise in the measured waveforms, correct identification of the shocks using this algorithm based on curvature was not always possible.

Holograms for the array operating in the CW regime were measured using a scanning tank setup, for three different beam configurations: (1) all array elements driven in phase so that only elevational focusing was provided by the lens, (2) with electronic azimuthal focusing along the axis of the probe at a focal length of Z_{F}=50 mm, henceforth referred to as the nominal focus location, and (3) with the beam focused at Z_{F}=50 mm and also steered to a lateral focal position of X_{F}32 10 mm. For these measurements, the Onda HGL-0200 hydrophone with AH-2020 20 dB preamplifier was connected to a 14-bit digitizer (Gage Razor 14, DynamicSignals LLC, Lockport, IL, USA), and a custom program written in Labview (NI, Austin, TX, USA) was used to control the positioner and acquire data. The Whittaker-Nyquist-Kotelnikov-Shannon sampling theorem requires that the spacing between hydrophone scan points be less than one-half of an acoustic wavelength in order for the hologram to represent all components of the propagating field without spatial aliasing artifacts. Holograms were measured at an array stimulus frequency of 1.071 MHz, with a sample spacing of 0.65 mm in both the X and Y directions such that the criteria for the sampling theorem were met.

Determining the magnitude and phase of the pressure at each hydrophone scan point was done by windowing the recorded pulse to a section of 5 cycles where CW conditions were well approximated and applying the fast Fourier transform (FFT) algorithm in Matlab. Directivity of the hydrophone was accounted for by computing the angular spectrum of the hologram using a 2D FFT and scaling the amplitude of each component of the resulting angular spectrum by a directivity factor based on the incidence angle upon the hydrophone. The holograms in the measurement plane were then backpropagated to reconstruct the source distribution at the face of the transducer using numerical evaluation of the Rayleigh integral.

[Par. No.] A high-speed imaging setup was used for direct observation of cavitation behavior in agarose gel phantoms. Agarose was used as the tissue-mimicking gel because of its ease of fabrication, optical transparency, low toxicity, and comparison with results of previous studies. The gel was prepared by adding agarose powder (UltraPure Agarose, Invitrogen, Waltham, MA, USA) to deionized (DI) water for an agarose/water concentration of 1.5% w/v. The solution was degassed by boiling in a microwave oven for 5 minutes and then immediately poured into a sample holder fabricated from acrylic plates and left to cool at room temperature for three hours. A corrugated absorber cast from silicone rubber was positioned in the bottom of the mold to reduce the effect of reverberations from the bottom of the sample holder. Once the agarose had polymerized, a layer of degassed water was added to the sample holder on top of the agarose to provide acoustic coupling between the dual- mode ultrasound probe and the agarose sample. The probe was suspended above the sample holder from a computer controlled 3-D positioner mounted above the workbench, while the sample holder with agarose was mounted on top of a manual linear positioning stage. This allowed the agarose to be moved relative to the HIFU focus so that a fresh region of the gel could be used for each exposure.

A Photron high-speed camera (model APS-RX, San Diego, CA) was used with a Carl Zeiss lens (model Makro-Planar 2/100 ZF, Thornwood, NY, USA) and bellows extension, located on one side of the sample holder. A 12-watt LED with collimating lens was positioned on the opposite side of the sample holder. To align the camera focus with the focus of the ultrasound beam, the agarose sample holder used for cavitation filming was replaced with a dummy sample holder with identical dimensions and with a 400-micron diameter wire positioned in the center. The probe was operated in the B-mode imaging regime to identify the location of the wire relative to the probe and to align to it. The lens focus and camera position were adjusted to provide an imaging resolution of 10 microns/pixel, based on the measured diameter of the wire target, for an overall field of view of 768×192 pixels (i.e., approximately 7.7×1.9 mm). For most of the acquired frames showing cavitation activity about the focus, the camera frame rate was 20 kfps with an exposure period of 4 μs. In addition to cavitation activity near the focus, a small number of images were captured with a larger field of view (1024×1024 pixels) and a frame rate of 3 kfps in order to determine the overall extent and distribution of the cavitation bubbles.

Each pHIFU exposure consisted of a single pulse at a stimulus frequency of 1.071 MHz, with a duration of 1 ms. Stimulus voltages were chosen to generate peak rarefactional pressures at the focus ranging from 1 MPa to 7 MPa, in 1 MPa increments, based on the saturation curves measured with the FOPH. For each value of stimulus pressure, three exposures were performed with corresponding camera frames. For each exposure, a trigger signal sent from the Verasonics to the camera initiated the acquisition of 25 image frames, with one image taken before the start of each pHIFU pulse for background subtraction. After each exposure, the agarose sample holder was moved 3 mm in the azimuthal direction using the linear stage, in order to position the pHIFU focus in a region of unexposed agarose in preparation for the next exposure.

Nonlinear simulations were performed using realistic source boundary conditions reconstructed from the measured acoustic holograms in order to compute the full transmitted pressure field in three dimensions at amplitudes required for generating cavitation. Directly measuring such a field with the FOPH would not only take a prohibitive amount of time but also could introduce errors at high shock amplitudes due to averaging over the effective fiber tip diameter, limited hydrophone bandwidth, and small positioning errors. Accurate nonlinear simulations are capable of providing a more realistic representation when shocks develop at the focus.

For each particular beam configuration simulated (i.e., beam focal length and steering angle) a boundary condition of specified acoustic pressure in the source plane z=0 was found by backpropagating the measured hologram from the measurement plane to the source plane using a combination of the Rayleigh integral and angular spectrum method. The numerically propagated pressure field was used to identify the central acoustic axis of the beam, and any small angular misalignment between the projected beam axis and the z axis of the numerical reconstruction grid was corrected for prior to reconstruction in the source plane. This was done to ensure the acoustic axis of the transducer coincided with the z axis of the computational domain.

[Par. No.] A formulation of the Westervelt equation convenient for beams propagating primarily along one axis was used for simulation of nonlinear acoustic fields transmitted by the dual-mode array over a range of output power levels. The equation accounts for the effects of diffraction, nonlinearity, and thermos-viscous absorption. Briefly, modeling forward propagation of the transmitted field, the Westervelt equation in a retarded coordinate system was used:

$$\frac{{\partial }^{2} p}{\partial z \partial τ} = \frac{{c}_{0}}{2} Δ p + \frac{β}{2 {ρ}_{0} {c}_{0}^{3}} \frac{{\partial }^{2} {p}^{2}}{\partial {τ}^{2}} + \frac{δ}{2 {c}_{0}^{3}} \frac{{\partial }^{3} p}{\partial {τ}^{3}} \qquad Equation 2$$

where p is the acoustic pressure, z is the spatial coordinate along the beam axis, τ=t−z/c_0 is the retarded time, t is the time, and Δ is the full three-dimensional Laplace operator. Physical properties for the propagation medium c₀, ρ₀, β, and δ are the small-signal sound speed, density, coefficient of nonlinearity, and sound diffusivity, respectively. Equation 2 was solved using the method of fractional steps, with an operator-splitting procedure of second-order accuracy: when marching over a grid step along the coordinate z, Equation 2 is divided into multiple simpler equations, which separate the operators for diffraction, nonlinearity, and absorption. The computed pressure field was represented in either the time domain or the frequency domain using a finite Fourier series expansion. The diffraction operator and absorption were both calculated in the frequency domain for each harmonic component of the pressure, while the nonlinear operator was calculated using two different methods. A frequency-domain approach was used at smaller axial distances where shock fronts were not yet formed, and a shock-capturing time-domain approach was used as the waveform steepness increased, and more harmonics were therefore required . . .

The imaging capabilities of the probe and the impact of different imaging parameters on overall image quality were investigated using both a commercial ultrasound quality assurance (QA) phantom (model 040GSE, CIRS Inc., Norfolk, VA, USA) and imaging liver and kidneys in vivo in an anesthetized pig. Beam generation and reconstruction of a B-mode image using the V-1 Verasonics system requires the specification of many acoustic and image reconstruction parameters. which influence the quality of the final image, including the center frequency of stimulus waveform, number of cycles in the pulse, number of transmitted beams used to reconstruct the image, the geometry of the transmitted beams (aperture, focal depth, beam angle, etc.), filter settings, and pulse timing. These imaging parameters were varied while viewing wire targets and hyperechoic inclusions in the CIRS QA phantom to optimize the image contrast and resolution at 30-70 mm depth range relevant to pancreatic tumor locations.

Once nominal imaging parameters had been determined, the resulting focal pressures for the transmitted beam were measured using the scanning tank setup of Section 2.B to determine the mechanical index (MI) in tissue as a function of source voltage. Finally, to demonstrate the suitability of the identified imaging parameters and capability for in vivo targeting centimeter-sized objects, B-mode images of the liver and kidneys of anesthetized pigs (n=3) used in experiments by another group were collected.

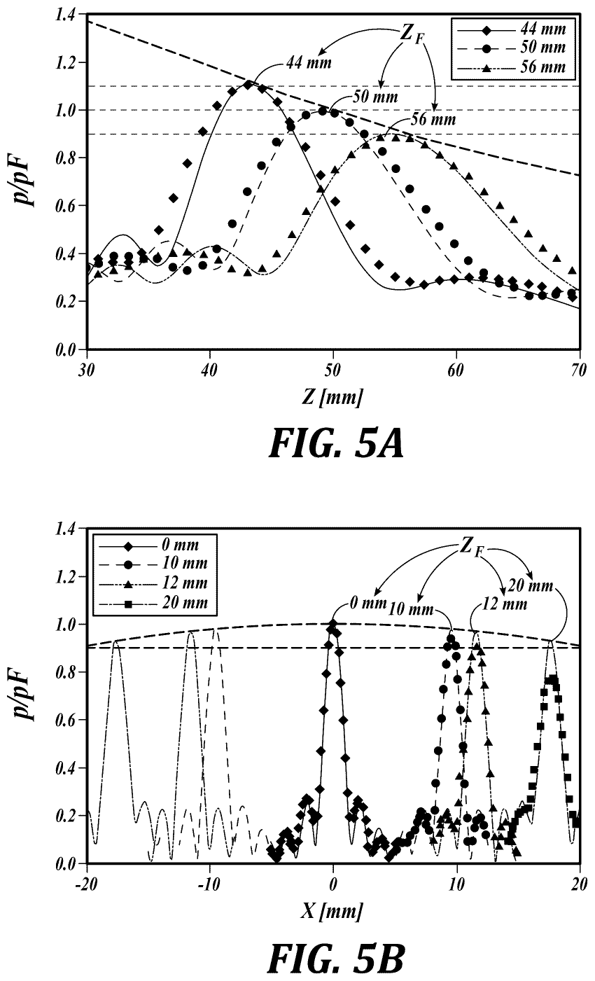

FIGS. 5A-5B show beam profiles, measured through the focus, when electronic phasing is used to focus the beam along the Z axis (a) and for azimuthal steering along the X axis (b), in accordance with the present technology. Lines indicate the beam profiles numerically simulated during probe design, while the measured beam profiles are indicated with the x markers. The pressure amplitudes have been normalized to the peak value at the nominal focus location, pF. Good agreement is seen in the axial profiles, while the measurements deviate from the simulated profiles as the steering angle is increased.

Measured beam profiles are shown below in FIGS. 5A-5B, along with the expected profiles based on ideal simulations in the linear operating regime, normalized to the peak pressure at the nominal focus position (i.e., Z_{F}=50 mm and X_{F}=0). FIG. 5A shows the beam profiles measured along the central axis of the probe when no azimuthal steering is used, for focal distances Z_{F} of 44 mm, 50 mm, and 56 mm. During the design of the probe, simulations in the linear regime were used to determine the steering limits of the beam, defined as the range of steered focus positions where the maximum pressure remains within a ±10% deviation from that for the nominal focusing position. This value was chosen such that the overall structure of the field would be similar in terms of beamwidth when steered in the linear regime while expecting full coverage of the intended targets. Based on the simulations, the expected axial steering limits were determined to be approximately ±6 mm from the nominal focus position, for a range in Z_{F} from 44 mm to 56 mm, and the azimuthal steering limits were determined to be X_{F}=±22 mm. The measured axial steering limits were found to agree well with those predicted by simulations, although the measured focal lobes were slightly longer along the acoustic axis of the beam. For circularly symmetric focused sources, pronounced pressure nulls are expected pre-focally and post-focally due to destructive interference between on-axis arrivals of the direct and edge wave. For rectangular sources, the lack of a single edge wave results in less interference and higher pressures in these nulls. Note that while the pressure amplitude gradually decreases when steering transversely from the focus and electronically focusing beyond the nominal focal length, it increases when moving the focus closer to the transducer array. The focusing gain of the transducer comprises two components: gain from focusing in the elevational plane provided by the fixed-length cylindrical lens, and gain from electronic focusing in the azimuthal plane. As the azimuthal focus is moved closer to the face of the transducer from the nominal focal length of 50 mm, the azimuthal gain increases. On the other hand, the two foci will no longer coincide. The increase in gain from reducing the azimuthal focal length is the dominating effect, owing to the smaller F-number for the azimuthal focusing, and the peak focal pressure increases as a result despite the difference in focal lengths.

Transverse beam profiles, when the beam was steered azimuthally with a nominal focal distance of Z_{F}=50 mm, are shown in FIG. 5B, along with beam profiles expected from the ideal simulations. It is seen that for small steering angles, measurements and simulations are in excellent agreement, while focal pressures are overestimated by the simulation as the steering angle is increased. The azimuthal steering limit was determined from the envelope of these profiles to be approximately X_{F}=±11.5 mm, significantly less than the limit of ±22 mm predicted by the ideal model. While only profiles in the positive X direction are shown in FIG. 5B, the steering limits were seen to be similar in the negative direction as well.

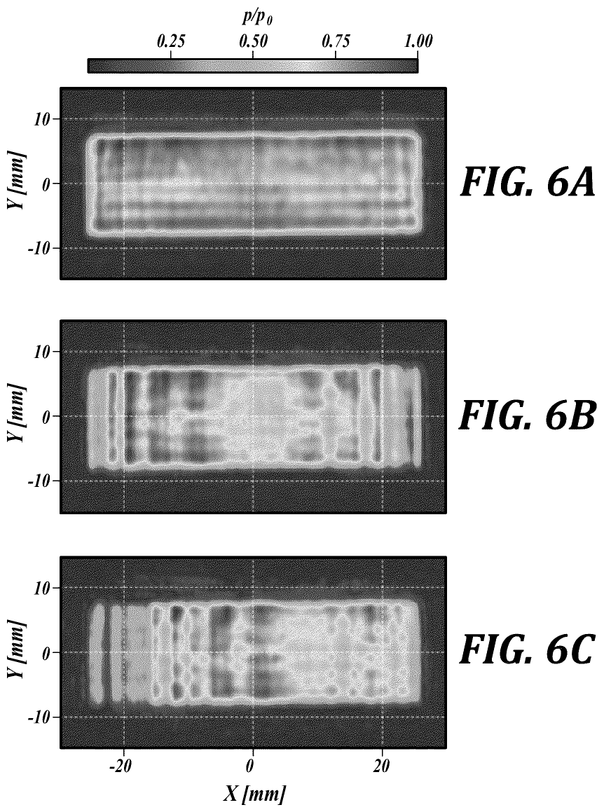

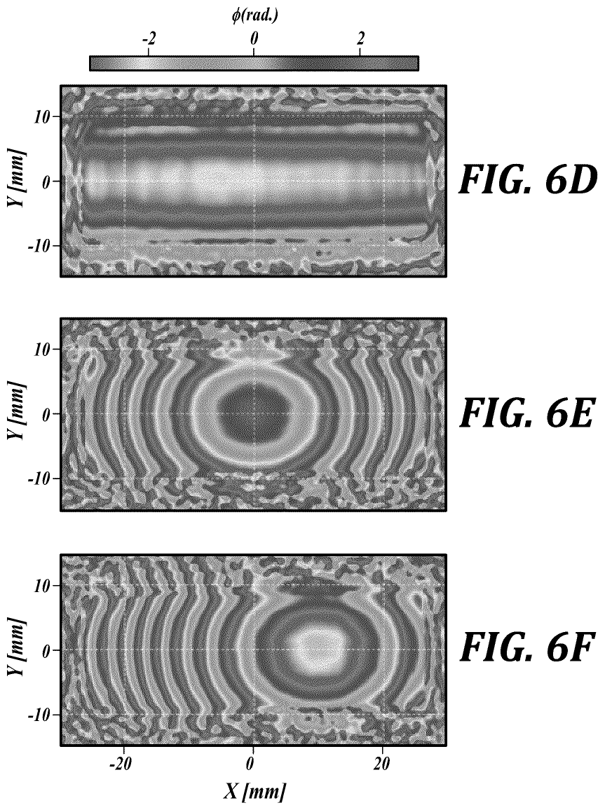

FIGS. 6A-6F show the source pressure magnitude and phase distributions, reconstructed from the measured holograms, when the array is configured for no electronic focusing (FIGS. 6A, 6D), on-axis focusing at 50 mm (FIGS. 6B, 6E), and focusing at 50 mm and steering towards 10 mm (FIGS. 6C, 6F), in accordance with the present technology. The effect of the cylindrical lens is clearly visible in the phase distribution when electronic focusing is disabled.

The source pressure magnitude and phase distributions, computed from the measured acoustic holograms, are shown in FIGS. 6A-6F for the following source configurations: all elements driven in phase (FIGS. 6A, 6D), elements phased for a focal length of Z_{F}=50 mm along the axis of symmetry (FIGS. 6B, 6E), and with the beam focused at Z_{F}=50 mm and steered to X_{F}=10 mm (FIGS. 6C, 6F).

Comparing the source magnitude and phase distributions for the three cases reveals several noteworthy features. For the case of uniform-phase excitation, the amplitude distribution is not perfectly uniform across the face of the array, but instead the distribution shows a region of higher magnitude near the upper left corner of the array. The variation of the phase distribution in the Y (elevational) direction is the effect of cylindrical focusing lens. However, some variation in phase along the X axis may be indicative of nonideal variations in lens thickness or bonding with the active elements, which may also be a cause of the areas with higher magnitude. Because the element size and pitch are smaller than the acoustic wavelength of λ=1.39 mm, the individual elements are not spatially resolvable in the source reconstruction but instead appear blended together.

The effects of adding electronic focusing and steering are both seen in the phase distributions, as expected, but also have a significant impact on the magnitude distribution across the face of the array. This variation in magnitude resulting from electronic focusing was not expected based on linear field simulations performed during the design of the probe). The source boundary condition used during the design-stage simulations assumed a uniform velocity magnitude distribution across the elements of the array and assumed that the elements were cylindrically curved in the Y direction to provide elevational focusing. One possibility is that the observed variation in magnitude across the transducer face is an artifact of the absorptive rubber lens used to provide elevational focusing for the fabricated array, rather than the cylindrically curved elements assumed in the simulations.

The accuracy of the source boundary conditions (BCs) reconstructed from the measured holograms was ascertained by computing the propagating field in the linear regime using the reconstructed BC as the source and comparing the resulting field with direct hydrophone measurements of the beam profiles. The profiles were seen to be in good agreement, with differences in the peak pressure less than 3%, well within the typical uncertainty of the hydrophone measurements. Azimuthal beam profiles for the nominal focusing case and for steering to X_{F}=10 mm are in close agreement. As a consequence of this agreement in the linear propagation regime, nonlinear simulations using these reconstructed boundary conditions were expected to provide an accurate representation of the field in the nonlinear propagation regime as well.

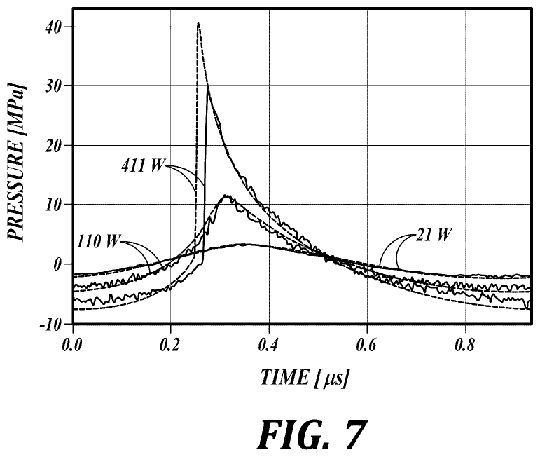

FIG. 7 is a graph of the comparison of measured focal pressure waveforms and nonlinear simulation results based on the reconstructed boundary condition for the nominal focus position, at three different stimulus levels, in accordance with the present technology. The waveforms are in good agreement when shocks are not present, while the hydrophone tends to measure a lower peak pressure once shocks form at the focus.

The measured and simulated nonlinear focal pressure waveforms are plotted in FIG. 7 at pulse-averaged acoustic power levels of 21 W, 110 W, and 411 W. The agreement is excellent up to 110 W, where the waveforms are nonlinearly distorted but without shocks. At the highest power of 411 W, however, peak positive pressure from simulation is higher than the measured value by more than 33%, whereas peak negative pressure is still in agreement within 7%. Large steep shock fronts are visible in both waveforms. Previous studies using diagnostic probes and therapeutic arrays have shown similar. The most likely reasons for discrepancies between simulated and measured focal waveforms that contain shocks are the spatial resolution of the FOPH, the accuracy with which the FOPH can be positioned in the center of the focus, and well as the bandwidth of FOPH limited by 100 MHz. At high power levels, the azimuthal beamwidth in terms of P+becomes comparable to the effective diameter of the fiber tip, so that the measured pressure is spatially averaged. As a result, the nonlinear simulations based on the reconstructed boundary conditions were expected to give a more accurate representation of the true nonlinear acoustic field near the focus.

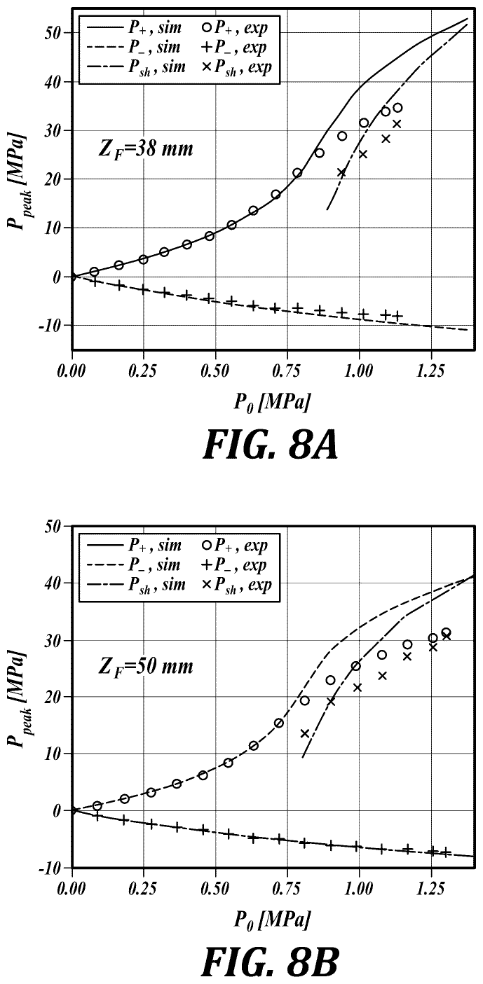

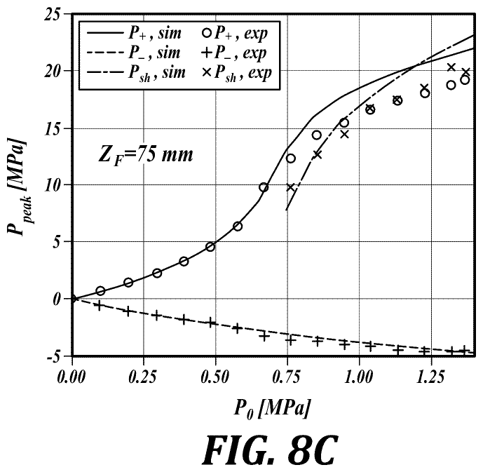

FIGS. 8A-8C show measured nonlinear focal pressures and shock amplitudes as a function of stimulus voltage for focal distances of 38 mm, 50 mm, and 75 mm without transverse beam steering compared with results from nonlinear simulation based on the holographic boundary conditions, in accordance with the present technology. At pressures above those required for shock development, the FOPH underestimates the peak positive pressure and corresponding shock amplitude.

The degree of focusing of a HIFU transducer, represented by its F-number, has a marked impact on the pressures required to generate nonlinearly distorted waveforms and shocks at the focus. Measured focal values of P₊ and P₋ are plotted in FIGS. 8A-8C as a function of effective source pressure, P₀, calculated using Eq. 1, for focal distances Z_{F} of 38 mm, 50 mm, and 75 mm without transverse beam steering. These focal distances correspond to azimuthal F-numbers of 0.74, 0.98, and 1.46, respectively. Also plotted is the shock amplitude, Pₛₕ, for those voltages sufficient for generating shocks at the focus. It is seen that the simulations and measurements are in excellent agreement in the quasilinear regime and for P₀ values below the threshold where shocks form in the simulated waveforms. Above this threshold, peak positive pressures measured using the FOPH, as well as the resulting shock amplitudes, are significantly lower than the simulation results, as was shown in the individual waveforms of FIG. 7.

The transmitted power, effective source pressure, and peak pressure values at the onset of shock formation are shown in Table 2. Because the FOPH measurements of focal pressure underestimate the true pressure once shocks form at the focus, P₊ and P₋ values are taken from the nonlinear simulations with holographic source BCs.

TABLE 2

| Z_{F} | W₀ | P₀ | P₋ | P₊ |
|---|---|---|---|---|
| [mm] | [W] | [kPa] | [MPa] | [MPa] |
| 38 | 203 | 890 | 8.0 | 30.2 |
| 50 | 165 | 803 | 5.6 | 21.3 |
| 75 | 141 | 743 | 3.1 | 13.0 |

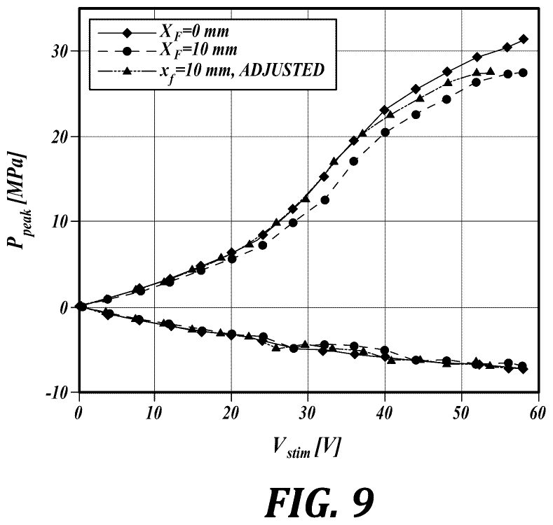

FIG. 9 shows peak pressures for the nominal steering configuration and for beam steering to XF=10 mm and ZF=50 mm, vs. applied stimulus voltage, in accordance with the present technology. For the “adjusted” curve, Vₛₜᵢₘ has been normalized by 1.08 in order to account for the difference in focal pressure in the linear regime when steering is employed (as shown in FIGS. 5B).

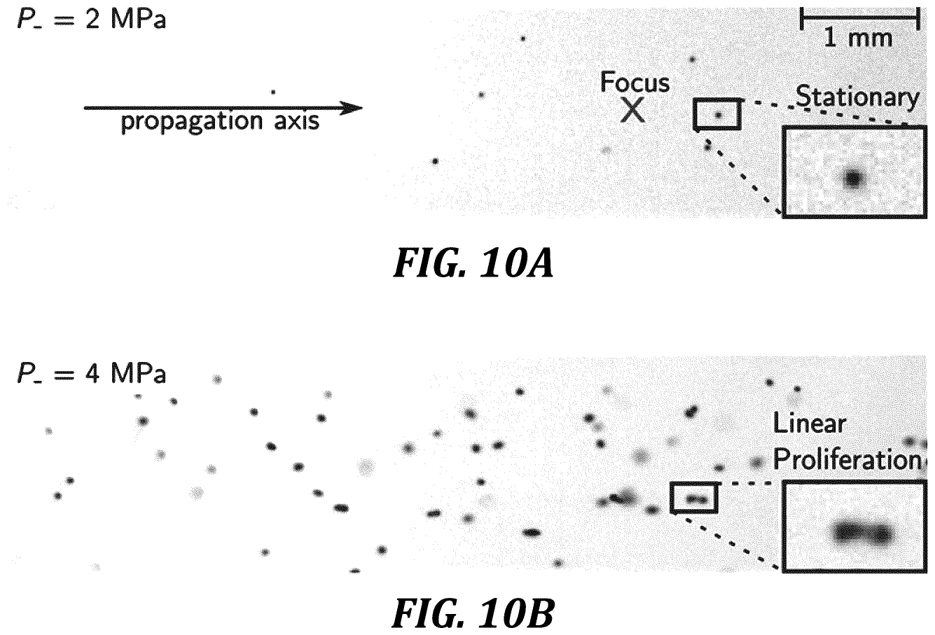

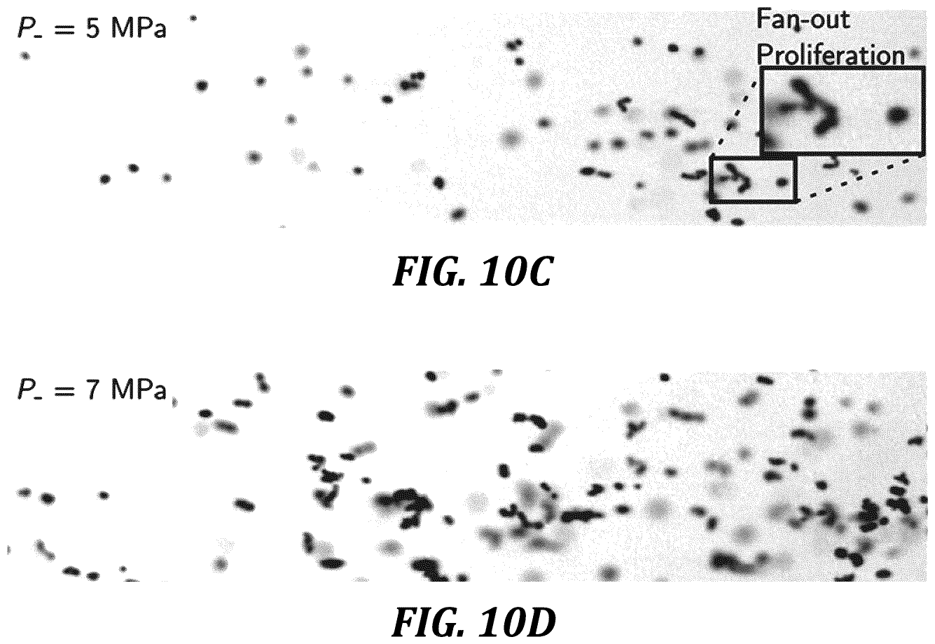

Maintaining the nonlinear field characteristics needed for cavitation generation when the beam is steered off-axis is critical to the therapeutic applications. FIG. 9 illustrates the measured peak focal pressures for Z_{F}=50 mm, both with and without off-axis steering to X_{F}=10 mm. Steering the beam azimuthally by 10 mm decreases the focal pressure, as expected, (see FIGS. 5A-5B), so that higher stimulus amplitudes are required to generate shocks at the focus as indicated in FIG. 10. However, when the voltage is scaled such that the same focal pressures are achieved with and without steering in the linear regime (a scale factor of about 1.08 for the voltage with steering), the peak pressure curves are in better agreement as seen in the blue “adjusted” curve. For this relatively small steering angle, the structure of the focal lobe in terms of its length and width is very similar to the nominal case. Therefore, when driven at voltages resulting in the same focal pressures in the linear regime, the nonlinear characteristics will be similar as well.

FIGS. 10A-10D are high-speed camera images showing the distribution of induced cavitation bubbles in the focal region, for on-axis beam focusing at ZF=50 mm, in accordance with the present technology. At P−=2 MPa, stationary cavitation bubbles are observed. The transition to proliferating behaviors occurred starting at a peak negative pressure of 4 MPa. Fan-out proliferation was seen for P− values at and above 5 MPa.

A series of high-speed camera frames showing induced cavitation bubble distributions near the focus and at the end of the 1 millisecond HIFU pulse are shown in FIGS. 10A-10D, for a range of focal P₋ values from 2 MPa to 7 MPa. No cavitation was observed in any camera exposures for a peak negative pressure of P₋=1 MPa. At P₋=2 MPa, isolated cavitation bubbles were observed and appeared stationary over the camera frames. When P₋ was increased to 4 MPa, a qualitative change in behavior was seen: with some of the cavitation bubbles initially isolated and stationary starting to split primarily along the direction of wave propagation into pairs and sets of bubbles, proliferating towards the transducer. This form of bubble proliferation behavior may also be referred to as linear proliferation. As the peak rarefactional focal pressure was increased to 5 MPa, bubbles were seen to proliferate laterally as well as axially, greatly increasing the number and spatial density of cavitation bubbles, which may be referred to as fan-out proliferation behavior. As the pressure increased further, this effect became even more pronounced, as seen in the image for P₃₁ =7 MPa. It should be noted that, in general, a combination of bubble behaviors was observed for pressures above the proliferation thresholds, where bubbles near the focus proliferated in a fan-out fashion, while those farther from the focus showed stationary cavitation behavior.

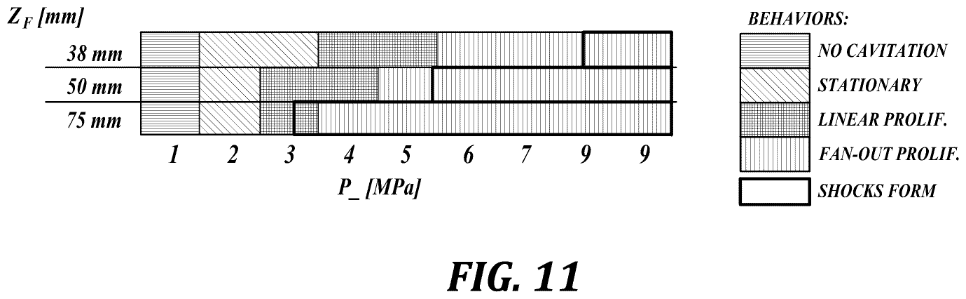

FIG. 11 shows categories of cavitation behavior observed in high-speed images for peak negative focal pressures between 1 MPa and 9 MPa, for on-axis focusing with focal lengths of 38 mm, 50 mm, and 75 mm, in accordance with the present technology.

Proliferation behaviors were categorized for on-axis focal distances of 38 mm, 50 mm, and 75 mm, as a function of P. from 1 MPa to 7 MPa in increments of 1 MPa. For a given combination of Z_{F} and P₋, FIG. 11 indicates the categories of proliferation that were observed. A particular cavitation behavior is indicated if it was seen occurring in at least one camera frame in at least one exposure at a given pressure. Also indicated in the figure are the ranges where shocks form at the focus, based on the nonlinear field simulations. While all three focal lengths were shown to induce stationary cavitation at P₃₁ =2 MPa, the transition to proliferating behaviors generally occurs at a smaller P₋ value as the focal distance increases, and for the cases of 50 mm and 75 mm the transition occurs at about the same pressure where focal shocks are seen to form.

A comparison of the observed bubble distributions with and without azimuthal beam steering to X_{F}=10 mm was recorded when the focal pressure in the on-axis case is P₋=6 MPa. In order to achieve the same peak negative focal pressure, the source amplitude was increased by 8% when beam steering was added. It was seen that the distribution of bubbles had the same general overall structure, and the transition from stationary to proliferating cavitation occurred near the same pressure threshold.

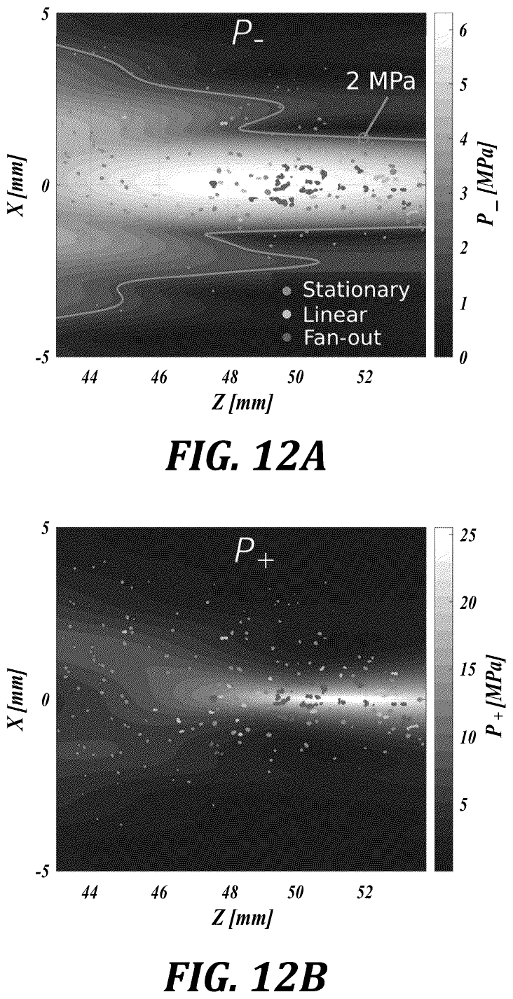

FIGS. 12A-12B are contour plots showing the peak negative pressure distribution (top) and peak positive (bottom) near the beam focus in the azimuthal plane, computed by nonlinear simulation with the source boundary condition measured by holography, in accordance with the present technology. The distribution of bubbles observed from high-speed photography for P₋=6 MPa is overlaid on each distribution and roughly agrees with the pressure distribution near the focus of the beam. The contour on the upper plot indicates the region where the pressure exceeds the cavitation threshold of 2 MPa.

The distribution of bubbles observed in the focal region can be compared with the pressure distribution in the same region, computed from nonlinear simulations based on the measured holograms. The resulting distributions for both peak positive and peak negative pressures are shown in FIGS. 12A-12B, for a peak negative focal pressure of P₋=6 MPa. The overall distribution of bubbles corresponds well with the region of the beam where P₃₁ exceeds the stationary cavitation threshold of about 2 MPa in the gel, as indicated by the blue contour in the top figure. The highest spatial density of bubbles was observed near the focus of the beam, where peak pressures are the greatest. It should be noted that while there were many bubbles outside of the nonlinear focal region in terms of P₊, most of the proliferating bubble behavior occurred near the region where the pressure waveforms exhibited significant nonlinear distortion. This reinforces the significant role nonlinearity can play in the transition from stationary to proliferating bubble behavior and is significant for application to tissue permeabilization where proliferating bubbles are expected to result in more tissue disruption than stationary bubbles. Even though bubbles are generated outside of the region where waveform nonlinearity is high, the localization of proliferating bubbles will result in a more localized effective response than would be implied by the overall size of the induced bubble distribution.

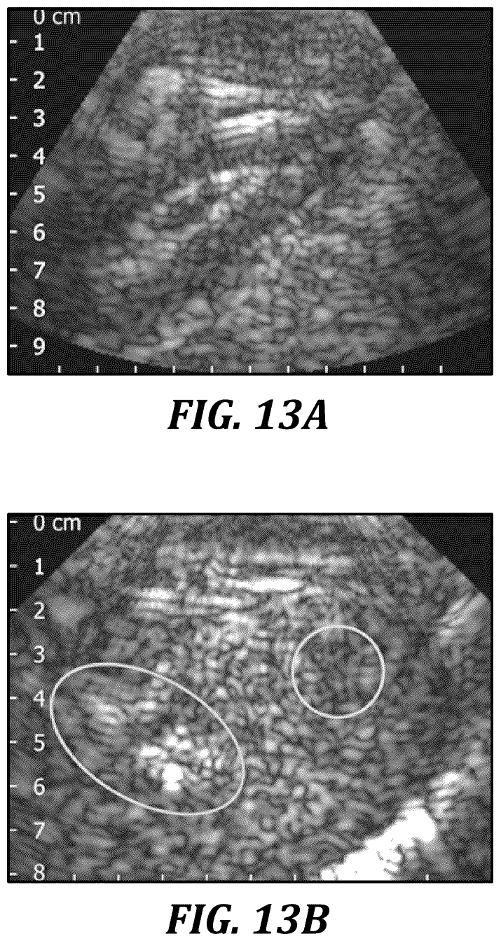

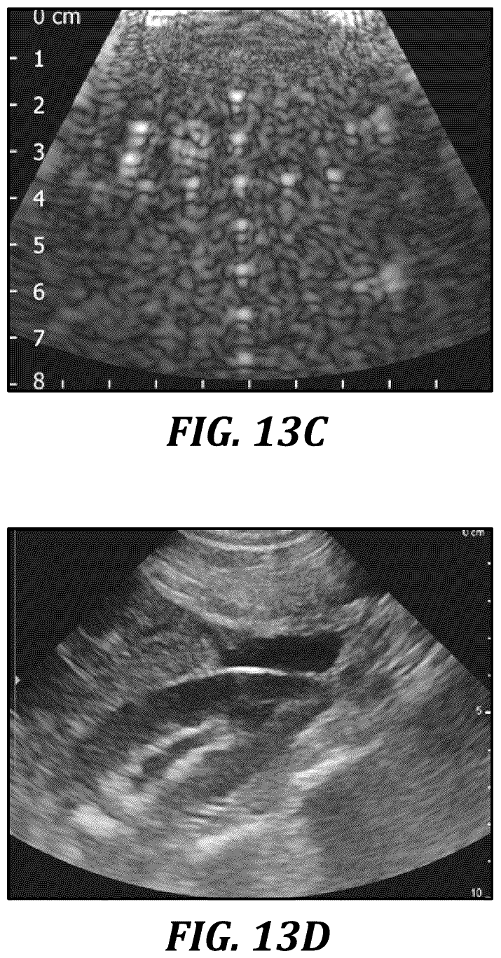

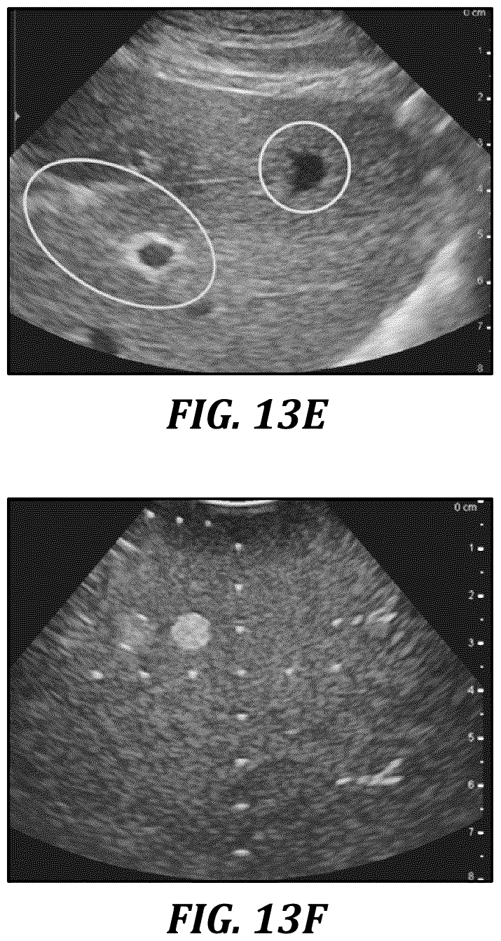

FIGS. 13A-13F are B-Mode images acquired with the dual-mode probe and Verasonics V-1 system at 1.5 MHz (FIGS. 13A-13C), compared with those collected using a 6C2s probe and BK3000 scanner at 2.5 MHz (FIGS. 13D-13F), in accordance with the present technology. Images were collected on a commercial QA phantom (FIGS. 13A, 13D), pig kidney (FIGS. 13B, 13E), and pig liver (FIGS. 13C, 13F).

Acceptable images were formed using 128 transmit beams, swept over an angle of 65 degrees, with the geometric focus set to a depth of 75 mm and F-number of 6.25. The array was operated in a pseudo-curvilinear mode, rather than the linear scan mode, in order to enlarge the field of view. Each transmitted pulse was two cycles in duration with a center frequency of 1.5 MHz, which is near the upper limit of the bandwidth of the probe. Images collected of three different targets using the dual-mode probe connected to the V-1 system are compared with those acquired with a commercial 6C2s diagnostic imaging probe in FIGS. 13A-13F. The 6C2s was connected to a BK3000 scanning system and was operated at a frequency of 2.5 MHz and an MI of approximately 1.4, while the MI for the dual-mode probe was approximately 0.40. The top and middle rows of FIGS. 13A-13F show images collected on a kidney and the liver, respectively, of two different anesthetized domestic farm pigs weighing between 45 kg and 50 kg. In both cases it was possible to clearly resolve centimeter-sized features, which could be used as anatomical landmarks for targeting during therapy. Images collected on the CIRS QA phantom are shown in the bottom row. The dual-mode probe was still capable of or resolving most of the small, embedded wire targets as well as the larger grayscale inclusions, while the 6C2s probe provided better image quality, with a smaller point spread and improved definition of the embedded targets. The lower operating frequency for the dual-mode probe resulted in a wider point-spread function, clearly seen in the point targets, as well as a larger characteristic size of the background speckle. Reverberations of the imaging pulse within the mechanical structure of the dual-mode probe initially resulted in a bright region covering a region approximately 1 cm to 2 cm from the face of the probe, and targets within 2 cm of the front of the probe were not resolvable. The TGC was set to the minimum possible level to eliminate this bright region at the top of the images collected with the dual-mode probe.

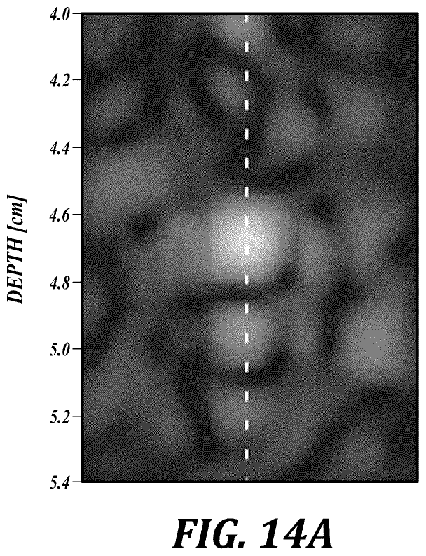

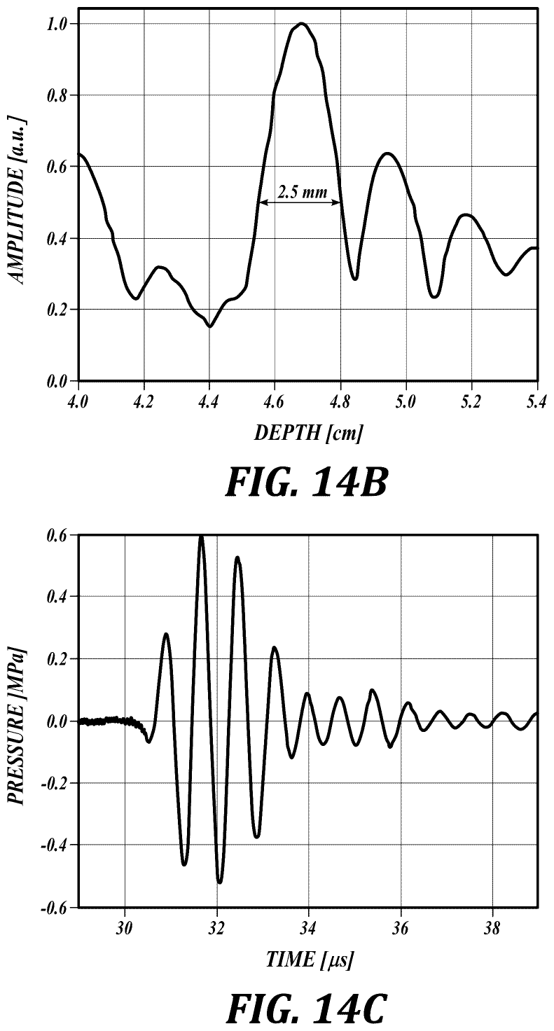

FIGS. 14A-14C are B-mode image of a single wire target in the CIRS phantom with a diameter of 100 μm and at an axial depth of approximately 4.7 cm (FIG. 14A), the intensity profile extracted through the center of the target image (FIG. 14B), and the pressure waveform measured at the focus of the imaging beam (FIG. 14C), respectively, in accordance with the present technology. The full width at half maximum (FWHM) diameter of the resolved target is 2.5 mm.

One measure of the resolution of an imaging system is the combined pulse-echo spatial impulse response of the system, also known as the point spread function. The apparent diameter of a small wire target in the CIRS phantom was used to estimate the point spread function. The intensity from the B-mode image shown in the lower left corner of FIGS. 13A-13F was extracted along a vertical line near the center of the image, through one of the wire targets at a depth of approximately 4.7 cm and is plotted in FIG. 14B. The FWHM diameter of the spot seen in the image is 2.5 mm, approximately 2.4 times the wavelength used for imaging. The focal pressure waveform for a single imaging pulse, measured using the 75 μm needle probe hydrophone is seen in FIG. 14C, used for calculating the MI corresponding to the chosen imaging parameters. It is seen that even though the transducer was driven with a stimulus voltage pulse consisting of two cycles, many more cycles are seen in the pressure waveform due to the finite bandwidth of the transducer.

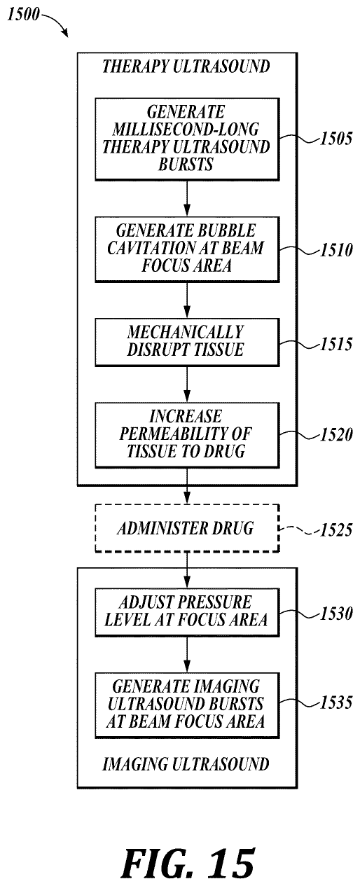

FIG. 15 is an example method 1500 of operating a dual phase probe as described herein, in accordance with the present technology. In some embodiments, the method 1500 is carried out by dual phase probe (such as dual phase probe 100) having a plurality of ultrasound elements (such as plurality of ultrasound elements 150A, 150B, 150C . . . 150N) configured to deliver both a therapy ultrasound and an imaging ultrasound. In some embodiments, the therapy ultrasound and imaging ultrasound are delivered sequentially by the dual phase probe 100. In some embodiments, the therapy ultrasound and the imaging ultrasound are emitted with same ultrasound emitting elements of the plurality of ultrasound emitting elements. In some embodiments, a first group of ultrasound emitting elements is configured for generating the therapy ultrasound, while a subsection of the first group of ultrasound emitting elements (such as subsection of ultrasound emitting elements 155) is configured for generating the imaging ultrasound (as shown in FIG. 4).

In block 1505, as part of the therapy ultrasound, millisecond-long therapy ultrasound bursts are generated. In some embodiments, the ultrasound bursts are nonlinearly distorted at a beam focus area of tissue. In some embodiments, the tissue comprises cellular membranes, collagen matrices, or glycosaminoglycan GAG-water complexes.

In block 1510, in response to the therapy ultrasound bursts, generating bubble cavitation at the beam focus area.

In block 1515, the tissue is manually disrupted by the bubble cavitation. In some embodiments, the tissue is disrupted non-thermally, that is, the tissue is not heated by the bubble cavitation.

In block 1520, by disrupting the tissue, permeability of the tissue to a drug administered to the subject is increased. In some embodiments, by increasing permeability, passive diffusion of the drug is also increased.

Optionally, in block 1525, a drug is administered. In some embodiments, the pFUS system is configured to administer the drug. In some embodiments, the drug is a chemotherapy drug.

In some embodiments, the dual-mode probe is further configured to operate as an imaging ultrasound probe.

In block 1530, pressure levels at the focus area are adjusted by adjusting parameters of the ultrasound probe. In some embodiments, the parameters include the power supply voltage amplitude and frequency, or varying the duty cycle of the stimulus waveform, or a pulse duration.

In block 1535, imaging ultrasound bursts are generated at the beam focus area of the tissue. In some embodiments, the dual-mode ultrasound probe is configured for generating the therapy ultrasound bursts and the imaging ultrasound bursts by same ultrasound emitting elements. In some embodiments, the imaging ultrasound bursts is B-mode imaging.

While illustrative embodiments have been illustrated and described, it will be appreciated that various changes can be made therein without departing from the spirit and scope of the invention.

What is claimed is:

1. A pulsed focused ultrasound (pFUS) system, comprising:
a dual mode ultrasound probe configured to generate therapy ultrasound bursts and generate imaging ultrasound bursts from a same one or more ultrasound emitting elements.

2. The pFUS system of claim 1, wherein the pFUS system is a dual phased array, and wherein the one or more ultrasound emitting elements is a plurality of ultrasound emitting elements.

3. The pFUS system of claim 2, wherein a first group of elements of the plurality of elements is configured for generating therapy ultrasound bursts, and wherein a subset of the first group of elements is configured for generating imaging ultrasound bursts.

4. The pFUS system of claim 2, wherein the pFUS system is configured for operating the dual-mode ultrasound probe as a therapy ultrasound probe by:
by the plurality of ultrasound emitting elements, generating millisecond-long therapy ultrasound bursts that are nonlinearly distorted at a beam focus area of a tissue;
in response to the therapy ultrasound bursts, generating bubble cavitation in the tissue at the beam focus area;
mechanically disrupting the tissue by the bubble cavitation;
by disrupting the tissue, increasing permeability of the tissue to a drug administered to the subject; and
by increasing permeability, enhancing passive diffusion of the drug.

5. The pFUS system of claim 4, wherein the pFUS system is configured for operating the dual-mode ultrasound probe as an imaging ultrasound probe by:
adjusting pressure levels at the focus area by adjusting parameters of the ultrasound probe; and
by the subset of the first group of elements, generating imaging ultrasound bursts at the beam focus area of the tissue.

6. The pFUS system of claim 4, wherein the tissue is disrupted non-thermally.

7. The pFUS system of claim 4, wherein the tissue comprises cellular membranes, collagen matrices, or glycosaminoglycan GAG-water complexes.

8. The pFUS system of claim 1, wherein the pFUS array system further comprises a controller, wherein the controller is configured for adjusting the power supply voltage amplitude and frequency, or varying the duty cycle of the stimulus waveform, or a pulse duration of the ultrasound therapy.

9. The pFUS system of claim 1, wherein the imaging ultrasound bursts are B-mode imaging.

10. A pulsed focused ultrasound (pFUS) array system for ultrasound imaging-guided treatment, comprising:
a multi-element linear array operable at a frequence range of 1-1.5 MHz and having an aperture with dimensions of approximately 14.8 mm×51.2 mm;
a cylindrical lens configured to focus the linear array to depth in tissue of approximately 50 mm; and
driving electronics with electronic beam steering capabilities and power levels configured to drive the linear array sufficient to achieve the focal pressures in tissue producing focal waveforms with shock fronts up to 45 MPa and peak negative pressures up to 9 MPa at focusing distances of 38-75 mm from the array.

11. A method for tissue treatment in a subject using pulsed focused ultrasound (pFUS), the method comprising:
operating a dual-mode ultrasound probe as a therapy ultrasound probe by:
generating millisecond-long therapy ultrasound bursts that are nonlinearly distorted at a beam focus area of the tissue;
in response to the therapy ultrasound bursts, generating bubble cavitation at the beam focus area;
mechanically disrupting the tissue by the bubble cavitation;
by disrupting the tissue, increasing permeability of the tissue to a drug administered to the subject; and
by increasing permeability, enhancing passive diffusion of the drug;
operating the dual-mode ultrasound probe as an imaging ultrasound probe by:
adjusting pressure levels at the focus area by adjusting parameters of the ultrasound probe; and
generating imaging ultrasound bursts at the beam focus area of the tissue,
wherein the dual-mode ultrasound probe is configured for generating the therapy ultrasound bursts and the imaging ultrasound bursts by same ultrasound emitting elements.

12. The method of claim 11, further comprising, administering the drug to the subject.

13. The method of claim 11, wherein the tissue is disrupted non-thermally.

14. The method of claim 11, wherein the tissue comprises cellular membranes, collagen matrices, or glycosaminoglycan GAG-water complexes.

15. The method of claim 11, further comprising adjusting the power supply voltage amplitude and frequency, or varying the duty cycle of the stimulus waveform, or a pulse duration.

16. The method of claim 11, further comprising administering chemotherapy drug to the patient after disrupting the tissue.

17. The method of claim 11, wherein the dual-mode ultrasound probe is a phased array probe, and wherein the dual-mode ultrasound probe is configured for:
generating the therapy ultrasound bursts by a first group of elements; and
generating the imaging ultrasound bursts by a subset of the first group of elements.

18. The method of claim 11, wherein the imaging ultrasound bursts is B-mode imaging.

19. The method of claim 11, wherein the pFUS transducer array system further comprises a targeting and cavitation monitoring component.

20. The method of claim 11, wherein the method further comprises:
imaging the tissue with an external imaging device before generating the therapy ultrasound bursts.
